(19) United States
(12) Patent Application Publication
(10) Pub. No.: US 2023/0085859 A1
(43) Pub. Date: Mar. 23, 2023

(54) IDENTIFICATION OF OPTIMAL RESOURCE ALLOCATIONS FOR IMPROVED RATINGS

(71) Applicant: Optum Services (Ireland) Limited, Dublin (IE)

(72) Inventors: Sean Carroll, Dublin (IE); Jacques Bellec, Dublin (IE); Ana Maria Pelaez, Letterkenny (IE); Kartik Asooja, Dublin (IE)

(21) Appl. No.: 17/478,702

(22) Filed: Sep. 17, 2021

(51) Int. Cl. G16H 40/20; G06Q 10/06; G06Q 40/08

(52) U.S. Cl. CPC G16H 40/20; G06Q 10/06393; G06Q 40/08

(57) ABSTRACT

Embodiments herein relate to resource allocation optimization. An example method includes receiving a resource allocation optimization request, the resource allocation optimization request comprising a plan identifier and a member data structure population identifier. The example method may further include retrieving a plurality of member data structures based at least in part on the member data structure population identifier. The example method may further include retrieving a plurality of measure data structures based at least in part on the plan identifier. The example method may further include, for each measure data structure of the plurality of measure data structures, generating an optimization score. Upon determining that a maximum obtainable benchmark points value meets a benchmark points value threshold, the example method may include generating a third number of benchmark points representing a required number of benchmark points for an overall rating level associated with the plan identifier to increase from a current rating level to a next rating level. The example method may further include generating a resource allocation optimization interface configured to render graphical representations of the plan identifier, the current rating level, the next rating level, the third number of benchmark points, and the plurality of measure data structures displayed in an order according to their respective optimization scores.

### BACKGROUND

Healthcare effectiveness data and information set (HEDIS) measures have been established to standardize performance measures for evaluating the quality of care provided by various health plans. That is, HEDIS measures are a comprehensive set of standardized performance measures designed to provide purchasers and consumers with information needed for reliable comparison of health plan performance. HEDIS performance data can be used to identify opportunities for improvement, monitor the success of quality improvement initiatives, track improvement, and provide standards that allow comparison with other plans.

Through applied effort, ingenuity, and innovation, many problems associated with the obtaining and use of HEDIS performance data have been solved by developing solutions that are included in embodiments of the present disclosure, many examples of which are described in detail herein.

### BRIEF SUMMARY

Embodiments herein relate to resource allocation optimization. An example method includes receiving a resource allocation optimization request, the resource allocation optimization request comprising a plan identifier and a member data structure population identifier. The example method may further include retrieving a plurality of member data structures based at least in part on the member data structure population identifier. The example method may further include retrieving a plurality of measure data structures based at least in part on the plan identifier. The example method may further include, for each measure data structure of the plurality of measure data structures, generating a first number of benchmark points associated with a first benchmark level, generating a second number of benchmark points based at least in part on a second number of compliant member data structures of the plurality of member data structures required for a second benchmark level that is higher than the first benchmark level, generating an optimization score.

Upon determining that a maximum obtainable benchmark points value meets a benchmark points value threshold, the example method may include generating a third number of benchmark points representing a required number of benchmark points for an overall rating level associated with the plan identifier to increase from a current rating level to a next rating level. The example method may further include generating a resource allocation optimization interface configured to render graphical representations of the plan identifier, the current rating level, the next rating level, the third number of benchmark points, and the plurality of measure data structures displayed in an order according to their respective optimization scores. The example method may further include providing the resource allocation optimization interface for display via display interface of a client computing device.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example resource allocation optimization computing entity in accordance with some embodiments discussed herein.

FIG. 3 provides an example client computing entity in accordance with some embodiments discussed herein.

FIG. 4 depicts examples of increasing compliance ratings, according to embodiments of the present disclosure.

FIG. 5 depicts examples of optimizing resource allocation to one or more measures, according to embodiments of the present disclosure.

FIG. 6 depicts examples of improving plan rating levels, according to embodiments of the present disclosure.

FIG. 7 depicts an example rating level determination process, according to embodiments of the present disclosure.

FIG. 8 depicts example data structure gathering and generation for use with embodiments of the present disclosure.

FIG. 9 depicts example rankings of measure data structures according to optimization scores, according to embodiments of the present disclosure.

FIG. 10 depicts examples of optimizing resource allocation to one or more measures, according to embodiments of the present disclosure.

FIG. 11 depicts an example resource allocation optimization interface for use with embodiments of the present disclosure.

FIG. 12 depicts an example data flow for implementing various embodiments of the present disclosure.

### DETAILED DESCRIPTION

Various embodiments of the present invention now will be described more fully hereinafter with reference to the accompanying drawings, in which some, but not all, embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout.

### I. Overview and Technical Improvements

HEDIS performance data can be used to identify opportunities for improvement, monitor the success of quality improvement initiatives, track improvement, and provide standards that allow comparison with other plans HEDIS ratings are determined by providers demonstrating that members received adequate care. HEDIS ratings are annual performance evaluations that take into account several clinical measures (e.g., ˜90 different clinical measures) HEDIS ratings enable improved healthcare for plan members as well as financial incentives and competitive advantages for those plan providers that reach higher plan ratings.

While HEDIS performance data can be used to identify opportunities for improvement, there are several constraints associated with obtaining appropriate (e.g., enough, high quality, etc.) data for evaluation, understanding weightings associated with different measures, and other criteria impacting a given measure's impact or potential impact on a rating. For example, data associated with a given member that may otherwise be used in determining a rating or an improvement may not actually be used because the member is not considered compliant. Not only is it computationally complex to identify those compliant member data structures, inclusion of non-compliant member data structures may make it impossible, due to lack of data, to determine a rating or an impact of a measure on an overall rating. In addition to requiring an understanding of what member data structures constitute compliant member data structures, conventional analyses require iterative and manual selection of measures to be assessed and do not provide relative comparisons of how much impact a given measure may have in relation to another measure.

Further, certain measures that are part of a rating may be of little impact on the overall rating, and as a result resources dedicated to implementing or improving those measures may be wasted or improperly utilized; instead, it may be preferable to determine which measures have the most impact on improving a rating for a given plan, so that resources dedicated to rating improvement can be conserved and conservatively expended. Determining which measures have the most impact on improving a rating also requires an understanding of the complexity of the criteria associated with a given measure, weightings associated with the criteria associated with a measure, weightings associated with the measure in the grand rating generation or valuation, distance to a next benchmark for a given measure, and what the remaining eligible population (e.g., member data structures) looks like.

Embodiments herein are directed to identifying optimal combinations of measures for improving HEDIS ratings. Embodiments herein balance and minimize the use of resources (e.g., computing, processing, communication, network, and the like) by identifying optimal allocation(s) of resources to maximize ratings while implementing or focusing resources on a minimal and optimal number of measures associated with the ratings.

Embodiments herein further generate an optimization score for each measure, where the optimization score represents a measure's ability to reach a next benchmark for a given plan. Each measure is further associated with possible points to be gained toward a given rating for a given plan such that the measures can be ranked according to one or more of the optimization score or possible points. Based upon the ranked presentation of measures, the minimum number of measures required for the plan to reach a next rating level may be selected.

Embodiments herein overcome challenges and drawbacks associated with conventional methods for evaluating measures and ratings because conventional methods are manual, involve subjectivity, and are not designed with improving ratings while minimizing the resources dedicated to maximizing the rating. Embodiments herein further provide for continuous monitoring and updating of suggested resources for dedication to different measures based upon updated or live data associated with eligible member data structures. The continuous monitoring and updating of suggested resource allocation enables systems to dedicate resources while data remains fresh and relevant. That is, a brute force approach may come to a solution but the time between obtaining the data upon which the solution is based and when the solution is determined may lead to the data being outdated or irrelevant.

### II. Definitions

As used herein, the terms “data,” “content,” “digital content,” “digital content object,” “information,” and similar terms may be used interchangeably to refer to data capable of being transmitted, received, and/or stored in accordance with embodiments of the present disclosure. Thus, use of any such terms should not be taken to limit the spirit and scope of embodiments of the present disclosure. Further, where a computing device is described herein to receive data from another computing device, it will be appreciated that the data may be received directly from another computing device or may be received indirectly via one or more intermediary computing devices/entities, such as, for example, one or more servers, relays, routers, network access points, base stations, hosts, and/or the like, sometimes referred to herein as a “network.” Similarly, where a computing device is described herein to transmit data to another computing device, it will be appreciated that the data may be sent directly to another computing device or may be sent indirectly via one or more intermediary computing devices/entities, such as, for example, one or more servers, relays, routers, network access points, base stations, hosts, and/or the like.

The terms “health plan,” “plan,” or “health care plan” refer to health insurance (or medical insurance), which is a type of insurance that covers the whole or a part of the risk of a person incurring medical expenses. Health care is the maintenance or improvement of health via the prevention, diagnosis, treatment, recovery, or cure of disease, illness, injury, and other physical and mental impairments in people. Health care is delivered by health professionals and allied health fields. Medicine, dentistry, pharmacy, midwifery, nursing, optometry, audiology, psychology, occupational therapy, physical therapy, athletic training and other health professions are all part of health care. It includes work done in providing primary care, secondary care, and tertiary care, as well as in public health.

The terms “health plan identifier,” “plan identifier,” or “health care plan identifier” refer to one or more items of data by which a health plan may be uniquely identified.

The term “rating level” refers to a programmatically generated value assigned to a health care plan reflective of a performance evaluation associated with the health care plan. An example rating level may be a HEDIS rating. The rating level may be based on a plurality of performance measures (also referred to herein as measures) across several domains of care. Examples of domains of care include effectiveness of care, access or availability of care, experience of care, utilization and risk adjusted utilization, health plan descriptive information, and measures collected using electronic clinical data systems.

The term “current rating level” refers to a rating level associated with a current or relatively recent timestamp for a given health care plan. That is, a given health care plan may be associated with a current rating level at a time of analysis regarding how to optimize allocation of resources to other combinations of resources in order to achieve a higher, or target rating level.

The term “target rating level” refers to a rating level that a given health care plan might achieve based on optimizing allocation of resources to combinations of various measures in accordance with embodiments herein. That is, a given health care plan may be associated with a current rating level at a time of analysis regarding how to optimize allocation of resources to other combinations of resources in order to achieve a higher, or target rating level, at a future timestamp.

The term “member” refers to a person to whom health care coverage or insurance has been extended by a policyholder or plan provider or any of their covered family members. Sometimes a member may be referred to as an insured or insured person.

The term “member identifier” refers to one or more items of data by which a member may be uniquely identified.

The terms “member data structure” or “member vector” refer to data structures containing a plurality of records (e.g., member vector records, member data structure records, member data records, member records, and the like), each containing an item of data associated with a member identifier which has an item identifier or name and an associated value. For example, a member data structure may contain a plurality of records where each record represents an item of health care related data associated with a member identifier. Each item of health care related data may be associated with a name and a value. A member data structure may also contain or be associated with a member identifier.

The terms “member vector record,” “member data structure record,” “member data record,” or “member record” refer to data structures within a member data structure or member vector for storing or organizing data associated with a given member identifier.

The terms “compliant member data structure” or “compliant member” refer to a member data structure associated with a member or member identifier that is considered compliant (e.g., who have had sufficient care) for a given measure identifier. For example, where a member has received sufficient care for a given measure, the member may be considered compliant for the given measure. Accordingly, a member data structure associated with a member identifier associated with the compliant member may be considered a compliant member data structure.

The terms “eligible member data structure” or “eligible member” refer to a member data structure associated with a member or member identifier that is considered eligible for a given measure identifier. For example, where a member qualifies for a given measure, the member may be considered eligible for the given measure. Accordingly, a member data structure associated with a member identifier associated with the eligible member may be considered an eligible member data structure.

The terms “non-compliant member data structure” or “non-compliant member” refer to a member data structure associated with a member or member identifier that is considered non-compliant (e.g., who have not had sufficient care) for a given measure identifier. For example, where a member has not received sufficient care for a given measure, the member may be considered non-compliant for the given measure. Accordingly, a member data structure associated with a member identifier associated with the non-compliant member may be considered a non-compliant member data structure.

The terms “ineligible member data structure” or “ineligible member” refer to a member data structure associated with a member or member identifier that is considered ineligible for a given measure identifier. For example, where a member does not qualify for a given measure, the member may be considered ineligible for the given measure. Accordingly, a member data structure associated with a member identifier associated with the ineligible member may be considered an ineligible member data structure.

The term “compliant member population” refers to a set of compliant member data structures from which data records may be used for scoring, evaluating, or optimizing a score or benchmark associated with a given measure identifier.

The term “measure” refers to a performance metric used in determining a rating level associated with a health care plan. The performance metric may include a set of technical specifications that define how a rating is calculated for a given quality indicator. Measure may be required to meet key criteria such as relevance, soundness and feasibility. A measure may be related to health care issues. Examples of measures may include antidepressant medication management, breast cancer screening, cervical cancer screening, children and adolescent access to primary care physician, children and adolescent immunization status, comprehensive diabetes care, controlling high blood pressure, prenatal and postpartum care, and more. A non-exhaustive list of example measures is included at the end of the present specification.

The term “measure identifier” refers to one or more items of data by which a measure may be uniquely identified.

The terms “measure data structure” or “measure vector” refer to data structures containing a plurality of records (e.g., measure vector records, measure data structure records, measure data records, measure records, and the like), each containing an item of data associated with a measure identifier which has an item identifier or name and an associated value. For example, a measure data structure may contain a plurality of records where each record represents an item of measure related data associated with a measure identifier. Each item of measure related data may be associated with a name and a value. A measure data structure may also contain or be associated with a measure identifier.

The terms “measure data record,” “measure vector record,” “measure data structure record,” or “measure record” refer to data structures within a measure data structure or measure vector for storing or organizing data associated with a given measure identifier.

The term “measure benchmark distance” refers to a range of values between a current benchmark points value associated with a given measure identifier and a targeted benchmark points value (e.g., a benchmark points value associated with achieving a next threshold or benchmark for the given measure identifier).

The term “measure complexity” refers to a varying attribute associated with a given measure that represents a level of difficulty associated with reaching compliance for the measure.

The term “measure compliance” refers to a programmatically generated value associated with how closely a defined number of measure data records of a measure data structure meet expected levels for a given measure identifier.

The term “measure numerator” refers to a number of compliant member data structures associated with a given measure identifier.

The term “measure denominator” refers to a number of eligible member data structures associated with a given measure identifier.

The term “measure rating” refers to a programmatically generated ratio of a measure numerator to a measure denominator.

The term “measure weighting value” refers to a numerical value applied (e.g., a weighting) to a measure data structure associated with a given measure identifier when a rating level is being determined based on a plurality of measure data structures.

The term “resource allocation optimization interface” refers a collection of graphical interface elements for rendering a representation of measure data structures and associated resource allocation optimization data and/or recommendations. The resource allocation optimization interface is configured for rendering via a display device of a computing device. The resource allocation optimization interface may be configured in accordance with constraints associated with the display device of the computing device (e.g., a size of the display device, an operating system of the computing device, a resolution of the display device, and the like). The resource allocation optimization interface may comprise a plurality of elements and/or panes configured for displaying the desired graphical representations in accordance with optimizing based on constraints associated with the display device.

### III. Computer Program Products, Methods, and Computing Entities

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

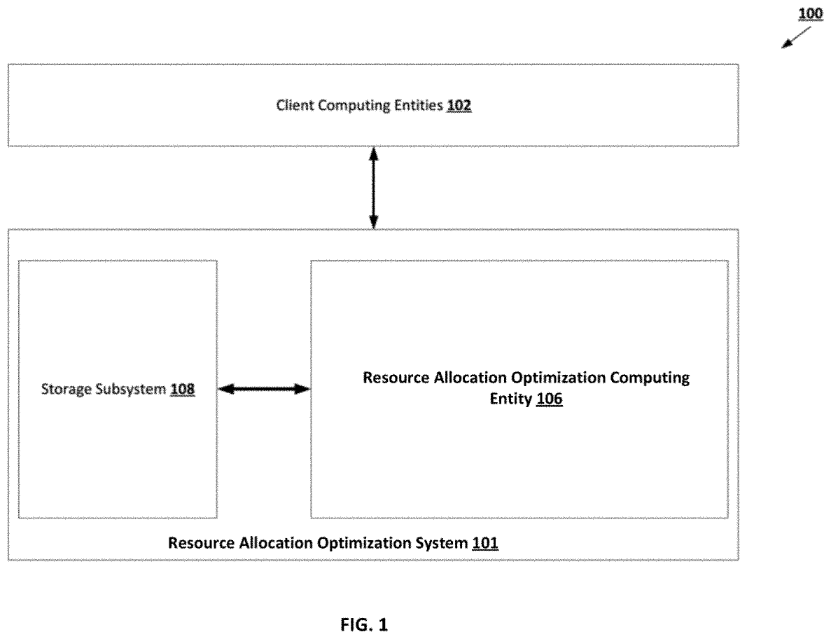

FIG. 1 is a schematic diagram of an example architecture 100 for performing resource allocation optimization. The architecture 100 includes a resource allocation optimization system 101 configured to receive resource allocation optimization requests from client computing entities 102, process the resource allocation optimization requests to generate resource allocation recommendations and provide the generated recommendations to the client computing entities 102, and automatically perform resource allocation-based actions based at least in part on the generated recommendations.

In some embodiments, resource allocation optimization system 101 may communicate with at least one of the client computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The resource allocation optimization system 101 may include a resource allocation optimization computing entity 106 and a storage subsystem 108. The resource allocation optimization computing entity 106 may be configured to receive resource allocation requests from one or more client computing entities 102 and process the resource allocation optimization requests to generate resource allocation recommendations corresponding to the resource allocation optimization requests, provide the generated recommendations to the client computing entities 102, and automatically perform resource allocation-based actions based at least in part on the generated recommendations.

The storage subsystem 108 may be configured to store input data used by the resource allocation optimization computing entity 106 to perform resource allocation optimization. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Resource Allocation Optimization Computing Entity

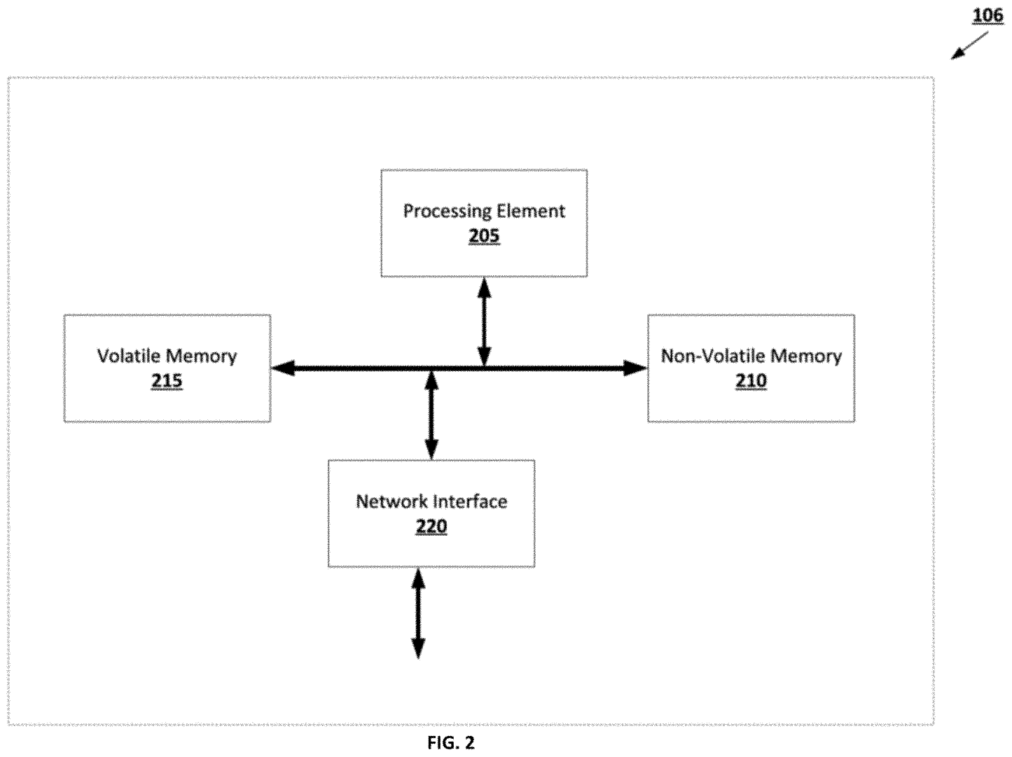

FIG. 2 provides a schematic of a resource allocation optimization computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the resource allocation optimization computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the resource allocation optimization computing entity 106 may include, or be in communication with, one or more processing elements 205 (also referred to as processors, processing circuitry, and/or similar terms used herein interchangeably) that communicate with other elements within the resource allocation optimization computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the resource allocation optimization computing entity 106 may further include, or be in communication with, non-volatile media (also referred to as non-volatile storage, memory, memory storage, memory circuitry and/or similar terms used herein interchangeably). In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity—relationship model, object model, document model, semantic model, graph model, and/or the like.

In one embodiment, the resource allocation optimization computing entity 106 may further include, or be in communication with, volatile media (also referred to as volatile storage, memory, memory storage, memory circuitry and/or similar terms used herein interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the resource allocation optimization computing entity 106 with the assistance of the processing element 205 and operating system.

As indicated, in one embodiment, the resource allocation optimization computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the resource allocation optimization computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the resource allocation optimization computing entity 106 may include, or be in communication with, one or more input elements, such as a keyboard input, a mouse input, a touch screen/display input, motion input, movement input, audio input, pointing device input, joystick input, keypad input, and/or the like. The resource allocation optimization computing entity 106 may also include, or be in communication with, one or more output elements (not shown), such as audio output, video output, screen/display output, motion output, movement output, and/or the like.

### Exemplary Client Computing Entity

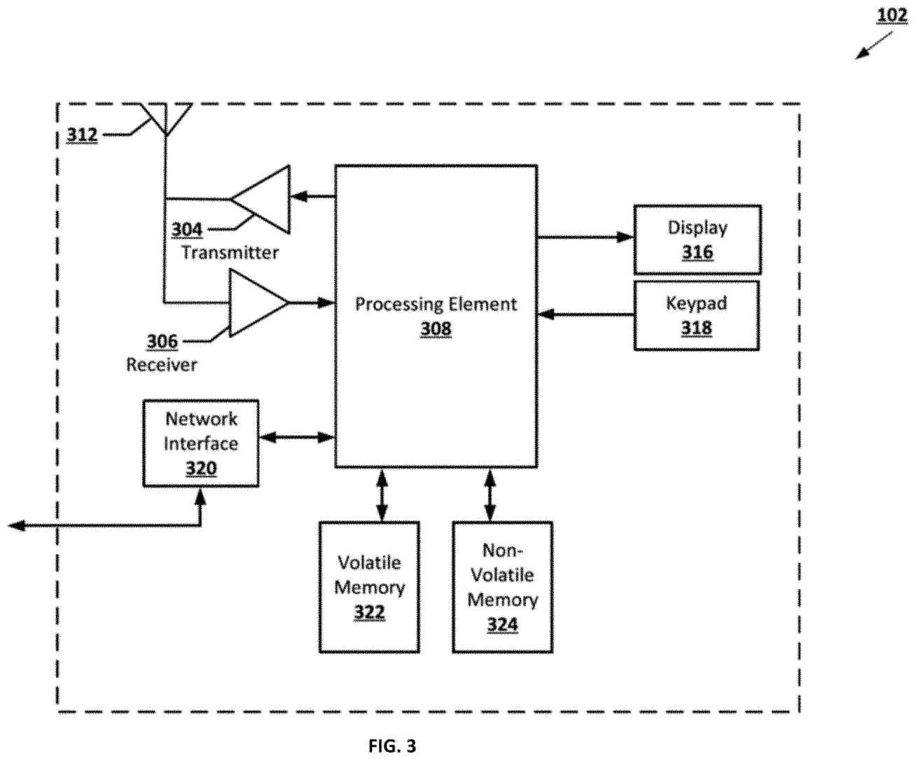

FIG. 3 provides an illustrative schematic representative of an client computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 102 can be operated by various parties. As shown in FIG. 3, the client computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 102 may be capable of operating with one or more air interface standards, communication protocols, modulation types, and access types. More particularly, the client computing entity 102 may operate in accordance with any of a number of wireless communication standards and protocols, such as those described above with regard to the resource allocation optimization computing entity 106. In a particular embodiment, the client computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 102 may operate in accordance with multiple wired communication standards and protocols, such as those described above with regard to the resource allocation optimization computing entity 106 via a network interface 320.

Via these communication standards and protocols, the client computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 102 can also download changes, add-ons, and updates, for instance, to its firmware, software (e.g., including executable instructions, applications, program modules), and operating system.

According to one embodiment, the client computing entity 102 may include location determining aspects, devices, modules, functionalities, and/or similar words used herein interchangeably. For example, the client computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 102 position in connection with a variety of other systems, including cellular towers, Wi-Fi access points, and/or the like. Similarly, the client computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the client computing entity 102 to interact with and/or cause display of information/data from the resource allocation optimization computing entity 106, as described herein. The user input interface can comprise any of a number of devices or interfaces allowing the client computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the client computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The client computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 102. As indicated, this may include a user application that is resident on the entity or accessible through a browser or other user interface for communicating with the resource allocation optimization computing entity 106 and/or various other computing entities.

In another embodiment, the client computing entity 102 may include one or more components or functionality that are the same or similar to those of the resource allocation optimization computing entity 106, as described in greater detail above. As will be recognized, these architectures and descriptions are provided for exemplary purposes only and are not limiting to the various embodiments.

In various embodiments, the client computing entity 102 may be embodied as an artificial intelligence (AI) computing entity, such as an Amazon Echo, Amazon Echo Dot, Amazon Show, Google Home, and/or the like. Accordingly, the client computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Exemplary System Operations

As described below, various embodiments of the present invention provide methods, apparatus, systems, computing devices, computing entities, and/or the like for optimizing allocation of resources in order to maximize ratings.

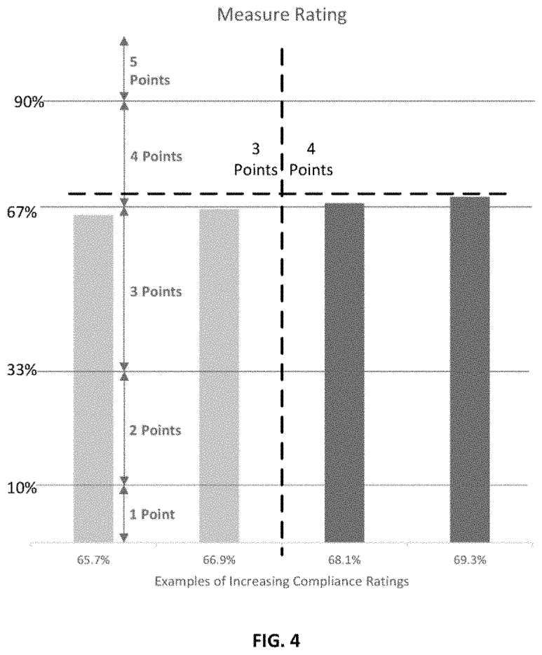

FIG. 4 depicts examples of increasing compliance ratings, according to embodiments of the present disclosure. In FIG. 4, a benchmark process involves identifying compliant member data structures and ensuring an adequate number of compliant member data structures are available for a plan or measure to reach a next benchmark level. That is, a measure rating level may be associated with a percentage of compliant member data structures available (e.g., 10%, 33%, 67%, and 90% in FIG. 4). Further shown in FIG. 4, points may be associated with a measure based on a measure's current benchmark. For example, a measure hitting just below a benchmark of 10% may be associated with 1 point. Reaching 33% from 10% may result in associating the measure with 2 points total (e.g., achieving above 10% but below 33% may result in associating the measure with 2 points total). Reaching just under 67% may result in the measure being associated with 3 points (e.g., achieving above 33% but below 67% may result in the measure being associated with 3 points total). Reaching above 67% but under 90% may result in the measure being associated with 4 points total, and reaching above 90% may result in the measure being associated with 5 points total. A healthcare plan may then be associated with a weighted average of the points associated with measures within the healthcare plan. Healthcare plans may be associated with half-point increments.

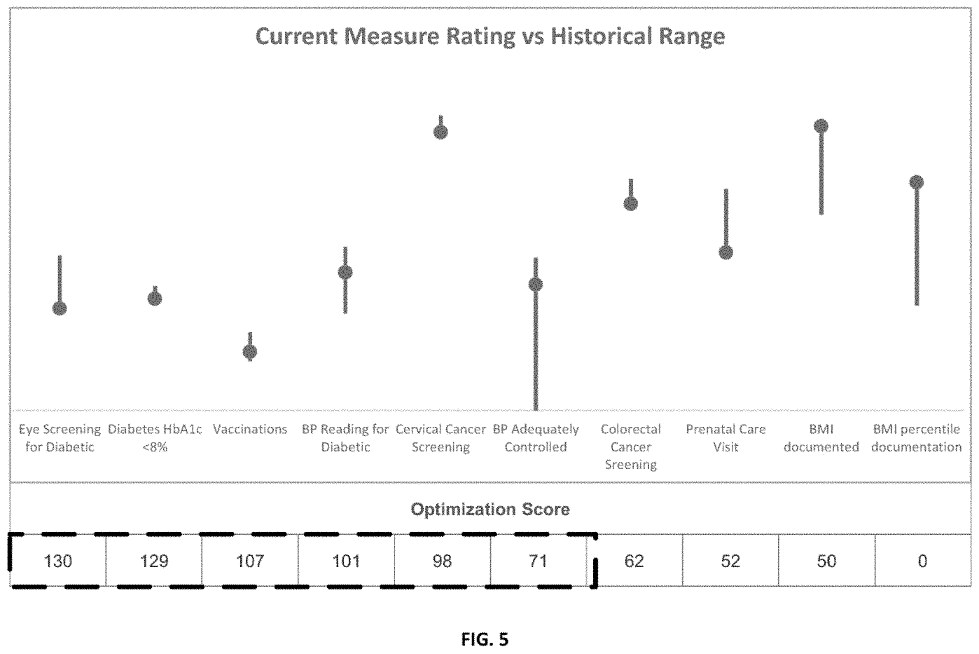

FIG. 5 depicts examples of optimizing resource allocation to one or more measures, according to embodiments of the present disclosure. In FIG. 5, a current measure rating (e.g., depicted by a dot in FIG. 5) for a given measure is depicted in relation to a historical range (e.g., depicted by a line associated with the dot in FIG. 5) for the given measure. Example measures in FIG. 5 include Eye Screening for Diabetic (e.g., with an optimization score of 130), Diabetes HbA1c <8% (e.g., with an optimization score of 129), Vaccinations (e.g., with an optimization score of 107), BP Reading for Diabetic (e.g., with an optimization score of 101), Cervical Cancer Screening (e.g., with an optimization score of 98), BP Adequately Controlled (e.g., with an optimization score of 71), Colorectal Cancer Screening (e.g., with an optimization score of 62), Prenatal Care Visit (e.g., with an optimization score of 52), BMI documented (e.g., with an optimization score of 50), and BMI percentile documentation (e.g., with an optimization score of 0).

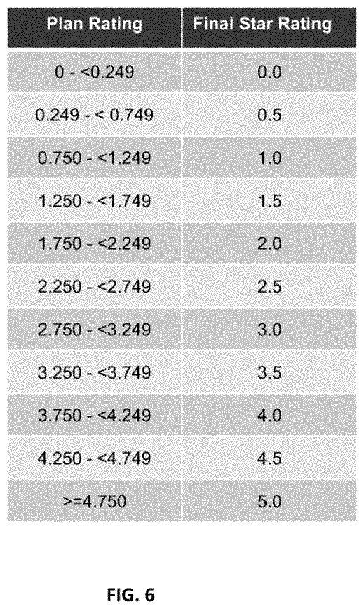

FIG. 6 depicts examples of improving plan rating levels, according to embodiments of the present disclosure. In FIG. 6, a table of plan ratings as compared to final star ratings is presented. It will be appreciated that, based on the example table in FIG. 6, by acquiring enough compliant members associated with a given measure, a health care plan may reach a next benchmark (e.g., a next or higher Plan Rating) which may improve the health care plans overall rating level (e.g., Final Star Rating). FIG. 6 depicts example thresholds for plan ratings relative to final star ratings.

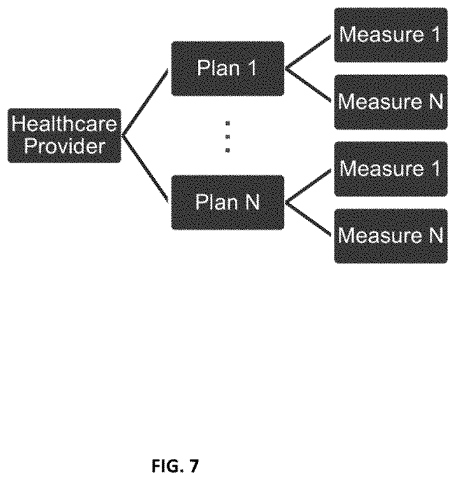

FIG. 7 depicts an example rating level determination process, according to embodiments of the present disclosure. In embodiments, a rating level associated with a health care plan (e.g., Plan 1, . . . Plan N) may be programmatically generated based on applying specific measure weighting values to each associated measure rating of a plurality of measure ratings associated with the health care plan. For example, in FIG. 7, Plan 1 may have a first rating level based in part on applying a first weighting value to Measure 1 and a second weighting value to Measure N. Plan N may have a second rating level based in part on applying a first (or other) weighting value to Measure 1 and a second (or other) weighting value to Measure N. In embodiments, a rating level associated with a health care provider (e.g., Healthcare Provider, in FIG. 7) may be programmatically generated based on applying specific weighting values to each health care plan rating level of a plurality of health care plans associated with the health care provider. For example, in FIG. 7, Healthcare Provider may have a rating level based in part on rating levels associated with Plan 1, . . . , and Plan N. The rating level for Healthcare Provider may be determined with or without applying weighting values to rating levels associated with any of its associated plans.

Embodiments herein optimize the above-described optimization analyses by including all measures in an evaluation of where resources should be allocated, as well as updating the analyses when measure data structures and member data structures are updated. A maximum measure rating may be considered for each measure data structure, and a plan rating may be generated based at least in part on the maximum measure ratings. The plan rating may increase or decrease toward a minimum percentage change required in order to meet a next star rating (e.g., see FIG. 6). Measure data structures may be ranked according to their ability to hit a next benchmark as well as their associated optimization scores.

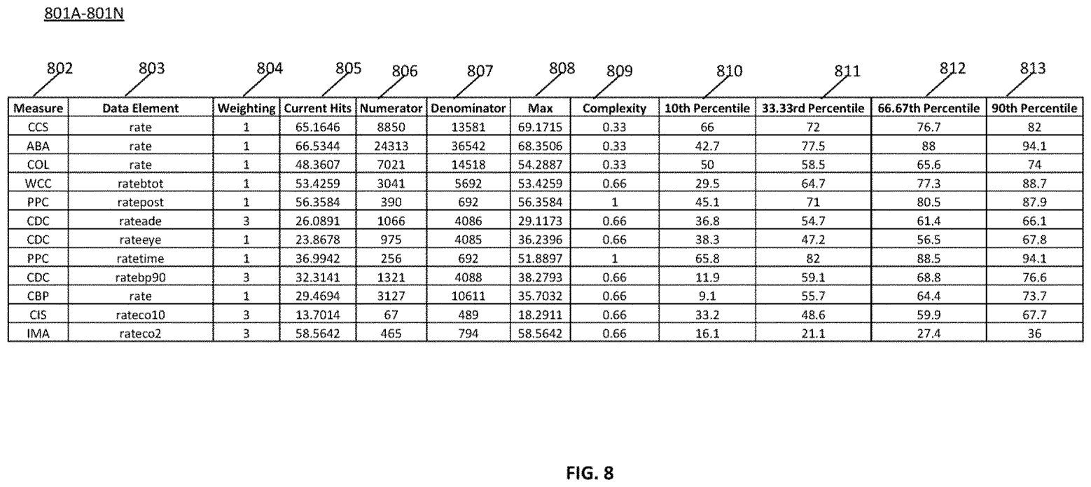

FIG. 8 depicts example data structure gathering and generation for use with embodiments of the present disclosure. In FIG. 8, a plurality of measure data structures or measure vectors (801A-801N) are depicted, where each measure vector or measure data structure is associated with a measure that is associated with a measure identifier (e.g., values in column 802). Each measure vector or measure data structure may be associated with a plurality of measure data records (e.g., 802-813) with associated values. For example, a first measure data structure may be associated with a measure identifier 802 of CCS, a Data Element 803 of “rate,” a Weighting 804 of “1,” Current Hits 805 of “65.1646,” a Numerator 806 of “8850,” a Denominator 807 of “13581,” a Max 808 of “69.1715,” Complexity 809 of “0.33,” 10th Percentile 810 of “66,” 33.33rd Percentile 811 of “72,” 66.67th Percentile 812 of “76.7,” and 90th Percentile 813 of “82.” It will be appreciated that the remaining measure data structures depicted in FIG. 8 follow a similar explanation as presented for the first measure data structure.

In FIG. 8, the data record associated with Current Hits 805 may represent a ratio of a first number of compliant member data structures available for the measure identifier (e.g., numerator as described above) to a second number of eligible member data structures available for the measure identifier (e.g., denominator as described above). Further, in FIG. 8, the data record associated with Max 808 (e.g., Max Hits) may represent a ceiling representing what is achievable for the given measure. That is, Max 808 may provide visibility into a range of possible improvement for a given measure data structure.

Based on the values in each measure data structure, a current benchmark as well as points reached (e.g., see FIG. 6) may be generated. Further, possible benchmarks to be reached are determined based on evaluating circumstances in which all maximum ratings were achieved (e.g., for all measure data structures). The maximum points to be reached, based on the possible benchmarks to be reached, are generated (e.g., see FIG. 6). If this improvement (e.g., the maximum points to be reached) is sufficient to move a plan's final star rating to the next star rating (e.g., see FIG. 6), a required percentage increase in the rating for achieving the next benchmark for each measure data structure is then generated. That is, for each measure data structure, a percentage increase in the measure rating is generated that represents how much the measure rating must increase in order for the measure to reach its next benchmark. From this, the required number of points for reaching the next star rating may be derived.

In embodiments, an optimization score for a measure data structure may be generated according to the following expression:

$$Optimization Score = \frac{Weighting * ( Max Hits - Current Hits )}{Complexity * ( \frac{Hits to Next Percentile}{Denominator - Numerator} )} \qquad ( 1 )$$

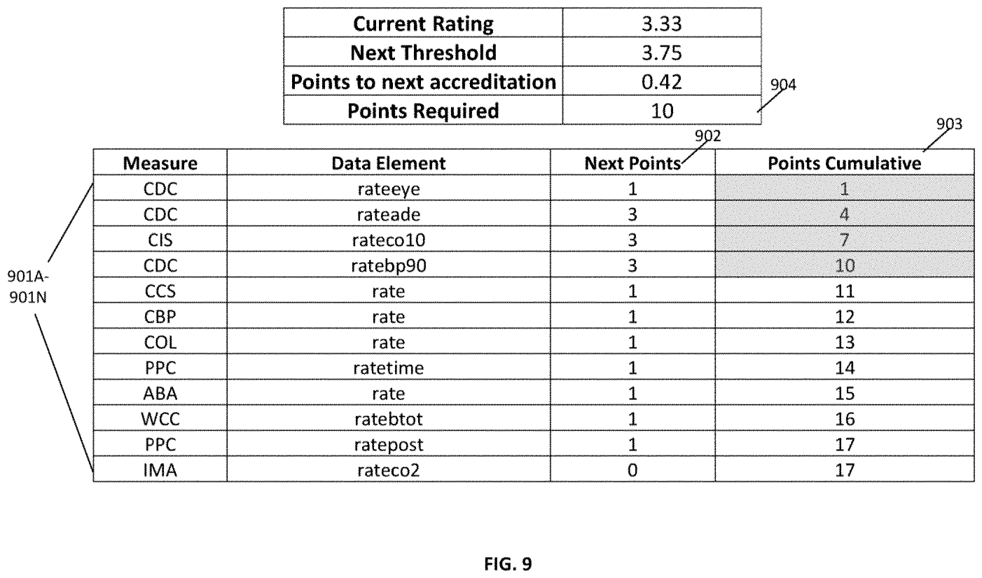

FIG. 9 depicts example rankings of measure data structures according to optimization scores, according to embodiments of the present disclosure. In FIG. 9, measure data structures 901A-901N are ranked according to optimization scores and then a number of points (e.g., Next Points 902) each measure data structure may be able to contribute to a health plan's overall star rating. Points Cumulative 903 provides visibility into the minimum number of measure data structures to which resources can be allocated in order to obtain the Points Required 904 to achieve a next rating level. In the example depicted in FIG. 9, Points Required 904 to the next star rating for the plan comprising measure data structures 901A-901N is 10. Accordingly, Points Cumulative 903 indicates that the required number of points may be obtained by allocating resources to measure data structures CDC/rateeye, CDC/rateade, CIS/rateco10, and CDC/ratebp90. In so doing, embodiments herein eliminate the need for allocating unnecessary resources to the remaining measure data structures, thereby saving on resources while maximizing ratings.

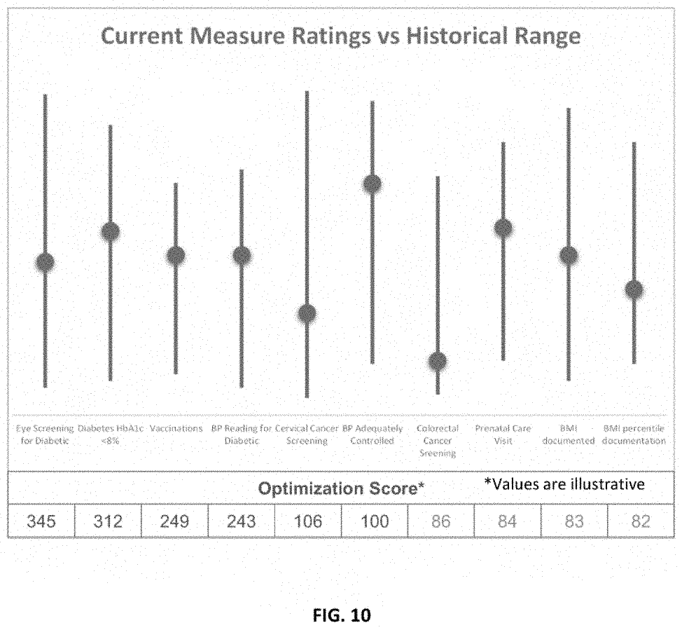

FIG. 10 depicts examples of optimizing resource allocation to one or more measures, according to embodiments of the present disclosure. In FIG. 10, a current measure rating (e.g., depicted by a dot in FIG. 10) for a given measure is depicted in relation to a historical range (e.g., depicted by a line associated with the dot in FIG. 10) for the given measure. Example measures in FIG. 10 include Eye Screening for Diabetic (e.g., with an optimization score of 345), Diabetes HbA1c <8% (e.g., with an optimization score of 312), Vaccinations (e.g., with an optimization score of 249), BP Reading for Diabetic (e.g., with an optimization score of 243), Cervical Cancer Screening (e.g., with an optimization score of 106), BP Adequately Controlled (e.g., with an optimization score of 100), Colorectal Cancer Screening (e.g., with an optimization score of 86), Prenatal Care Visit (e.g., with an optimization score of 84), BMI documented (e.g., with an optimization score of 83), and BMI percentile documentation (e.g., with an optimization score of 82).

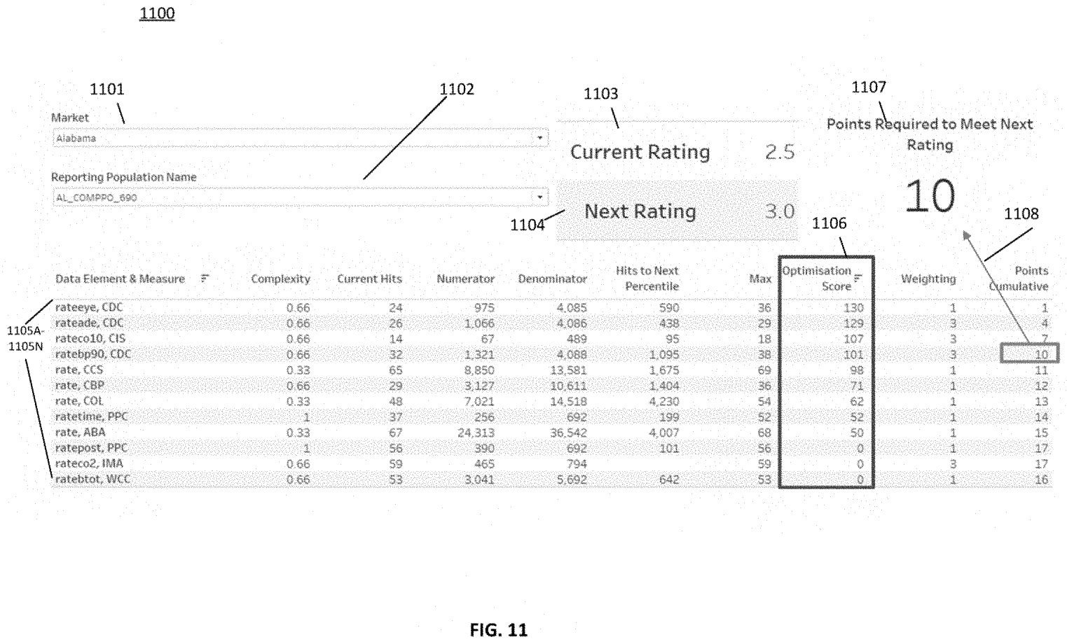

FIG. 11 depicts an example resource allocation optimization interface 1101 for use with embodiments of the present disclosure. In embodiments, a resource allocation optimization interface 1101 may be configured to render a plan selection element (not shown) for selecting a specific health care plan for which a resource allocation optimization analysis may be performed. In embodiments, the resource allocation optimization interface 1101 may be configured to render a first selection element 1101 (e.g., for selecting a “Market” in FIG. 11; e.g., the “Market” in FIG. 11 is depicted as “Alabama”). The interface 1101 may be further configured to render a second selection element 1102 (e.g., for selecting a “Reporting Population Name” in FIG. 11; e.g., the “Reporting Population Name” in FIG. 11 is “AL_COMPPO_690”). In embodiments, the second selection element 1102 may be for selecting a plurality of member data structures for use in generating a resource allocation optimization analysis and recommendation. That is, in FIG. 11, the “Reporting Population Name” of “AL_COMPPO_690” may comprise a plurality of member data structures. The interface 1101 may further be configured to render a “Current Rating” 1103 element (e.g., 2.5 in FIG. 11) representing a current rating associated with the selected plan, as well as a “Next Rating” 1104 element representing a rating that may be obtained by or associated with the plan if resource allocation were to be adjusted. The interface 1101 may further be configured to render a “Points Required to Meet Next Rating” element 1107 representing a number of points, generated according to embodiments herein associated with FIGS. 6-9), required for the plan to achieve the next rating level.

The resource allocation optimization interface 1101 is further configured to render a plurality of measure data structures 1105A-1105N displayed in an order ranked according to their respective optimization scores 1106. Each rendered measure data structure is also associated with points the measure data structure may be associated with if resources are allocated to the measure data structure, and Points Cumulative displays a rendering of cumulative points, starting from the highest ranked optimization score, such that a user may witness visualization of selecting the minimum number or combination of measure data structures to which resources may be allocated in order to achieve a next rating level. Accordingly, the interface 1101 indicates that the required number of points may be obtained by allocating resources to measure data structures CDC/rateeye, CDC/rateade, CIS/rateco10, and CDC/ratebp90. In so doing, embodiments herein eliminate the need for allocating unnecessary resources to the remaining measure data structures, thereby saving on resources while maximizing ratings.

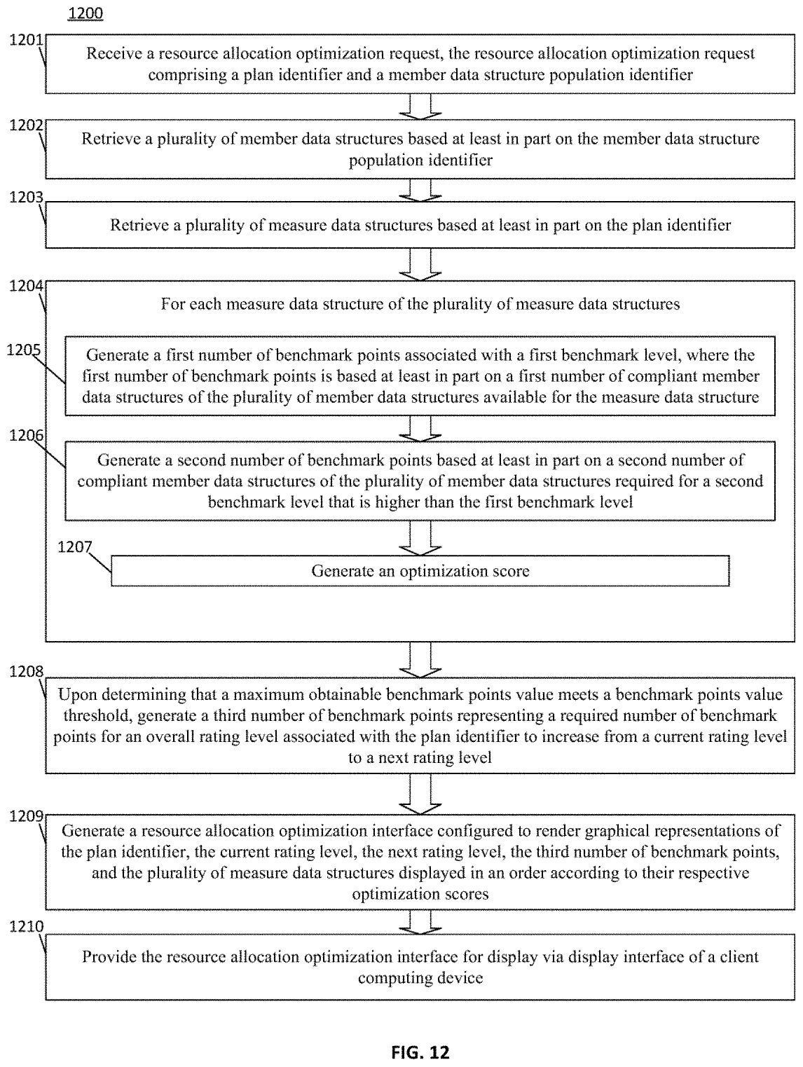

FIG. 12 depicts an example data flow 1200 for implementing various embodiments of the present disclosure. In embodiments, an example data flow 1200 includes receiving 1201, for example by a resource allocation optimization computing entity 106, a resource allocation optimization request. In embodiments, the resource allocation optimization request comprises a plan identifier and a member data structure population identifier.

In embodiments, the example data flow 1200 further includes retrieving 1202, for example by a resource allocation optimization computing entity 106 and from a data repository according to the present disclosure, a plurality of member data structures based at least in part on the member data structure population identifier.

In embodiments, the example data flow 1200 further includes retrieving 1203, for example by a resource allocation optimization computing entity 106 and from a data repository according to the present disclosure, a plurality of measure data structures based at least in part on the plan identifier.

In embodiments, the example data flow 1200 further includes, for example by a resource allocation optimization computing entity 106 and for each measure data structure of the plurality of measure data structures 1204, generating 1205 a first number of benchmark points associated with a first benchmark level. In embodiments, the first number of benchmark points is based at least in part on a first number of compliant member data structures of the plurality of member data structures available for the measure data structure.

In embodiments, the example data flow 1200 further includes, for example by a resource allocation optimization computing entity 106 and for each measure data structure of the plurality of measure data structures 1204, generating 1206 a second number of benchmark points based at least in part on a second number of compliant member data structures of the plurality of member data structures required for a second benchmark level that is higher than the first benchmark level.

In embodiments, the example data flow 1200 further includes, for example by a resource allocation optimization computing entity 106 and for each measure data structure of the plurality of measure data structures 1204, generating 1207 an optimization score.

In embodiments, the example data flow 1200 further includes, for example by a resource allocation optimization computing entity 106, upon determining that a maximum obtainable benchmark points value meets a benchmark points value threshold, generating 1208 a third number of benchmark points representing a required number of benchmark points for an overall rating level associated with the plan identifier to increase from a current rating level to a next rating level.

In embodiments, the example data flow 1200 further includes, for example by a resource allocation optimization computing entity 106, generating 1209 a resource allocation optimization interface configured to render graphical representations of the plan identifier, the current rating level, the next rating level, the third number of benchmark points, and the plurality of measure data structures displayed in an order according to their respective optimization scores.

In embodiments, the example data flow 1200 further includes, for example by a resource allocation optimization computing entity 106, providing 1210 the resource allocation optimization interface for display via display interface of a client computing device. In embodiments, providing an interface for display may include transmission of the interface to the client computing device. In embodiments, providing an interface for display may include locally providing the interface for display. In embodiments, providing an interface for display may include causing display of the interface via the display interface.

In embodiments, the resource allocation optimization interface is further configured to render a graphical representation of the second number of benchmark points associated with each measure data structure. In embodiments, the resource allocation optimization interface is further configured to render an indication of a minimum number of measure data structures to which resources should be allocated in order to achieve the next rating level. In embodiments, the resource allocation optimization request is received originating from the client computing device.

In embodiments, the plan identifier and member data structure population identifier are received as a result of electronic interactions with a graphical user interface by a user of the client computing device.

In embodiments, the current rating level is a HEDIS rating. In embodiments, the next rating level is a HEDIS rating.

In embodiments, the optimization score is generated according to:

$$Optimization Score = \frac{Weighting * ( Max Hits - Current Hits )}{Complexity * ( \frac{Hits to Next Percentile}{Denominator - Numerator} )}$$

where Weighting represents a weighting value associated with a measure identifier of a measure data structure for which the optimization score is being generated, Max Hits represents a maximum number of compliant member data structures available for the measure identifier, Complexity represents a complexity value associated with the measure identifier, Numerator represents a first number of compliant member data structures available for the measure identifier, Denominator represents a second number of eligible member data structures of the plurality of member data structures available for the measure identifier, Current Hits represents a ratio of Numerator to Denominator, and Hits to Next Percentile represents a third number of required additional compliant member data structures in order to achieve a next percentile for the measure identifier.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### VII. Example Measures

A non-exhaustive list of example measures is included below.

| Measure | Care, screening or test |
|---|---|
| Well-child visits in the first 30 | Six or more well-child visits (Well-Care Value Set) on |
| months of life | different dates of service on or before the 15-month birthday. |
| | Two or more well-child visits on different dates of service |
| | between the child's 15-month birthday plus 1 day and the 30- |
| | month birthday. |
| | The well-child visit must occur with a PCP, but the PCP does |
| | not have to be the practitioner assigned to the child. |
| Cardiac rehabilitation | 18 years and older who attended cardiac rehabilitation |
| | following a qualifying cardiac event, including myocardial |
| | infarction, percutaneous coronary intervention, coronary artery |
| | bypass grafting, heart and heart/lung transplantation or heart |
| | valve repair/replacement. |
| Kidney health for patients with | The percentage of members 18-85 years of age with diabetes |
| diabetes | (type 1 and type 2) who received a kidney health evaluation, |
| | defined by an estimated glomerular filtration rate |
| | (eGFR) and a urine albumin-creatinine ratio (uACR). |
| Osteoporosis screening in older | Women 65-75 years of age who received osteoporosis |
| women | screening |

Children and adolescents

| | |
|---|---|
| Weight assessment and | Patients 3-17 who had evidence of body mass index (BMI) |
| counseling for nutrition and | percentile including height and weight, counseling or referral |
| physical activity for children | for nutrition or indication nutrition was addressed. Counseling |
| and adolescents. | or referral for physical activity or indication physical activity |
| Ages 3-17 years | was addressed during an outpatient visit either by a claim or as |
| | a medical record entry. |
| Child and adolescent well-care | Annual well-care visit with a PCP or OB/GYN. |
| visits | |
| Ages 3-21 years | |
| Childhood immunization | 4 DTaP (none prior to 42 days of age) |
| Series must be completed by the | 3 IPV (none prior to 42 days of age) |
| second birthday | 1 MMR or documented history of illness or a |
| | seropositive test occurring prior to second birthday |
| | 3 HiB (none prior to 42 days of age) |
| | 3 HEP B or documented history of illness or a |
| | seropositive test occurring prior to second birthday |
| | 1 HEP A or documented history of illness or a |
| | seropositive test occurring prior to second birthday |
| | 1 VZV, or documented history of illness or a |
| | seropositive test occurring prior to second birthday |
| | 2-3 rotavirus (none prior to 42 days of age) |
| | 2 influenza (Added live attenuated influenza vaccine |
| | (LAIV) |
| Immunizations for adolescents | 1 meningococcal vaccine on or between the member's |
| 13 year-olds | 11th or 13th birthday (“meningococcal conjugate |
| | vaccine” or “meningococcal polysaccharide vaccine” |
| | meet criteria) |
| | 1 Tdap or 1 Td on or between the member's 10th or 13th |
| | birthday |
| | 3 doses of HPV vaccine with different dates of service, |
| | on or between the ninth and 13th birthday. |
| | OR |
| | 2 doses of HPV with different dates of service and at |
| | least 146 days between doses on or between the ninth |
| | and 13th birthdays. |
| | Exclusions: anaphylactic reaction to the vaccine or its |
| | components |
| Lead screening in children | Children who received at least one capillary or venous lead |
| (Medicaid only) | screening test on or before their second birthday. |
| Children aged 2 | |
| Treatment of children with | Children three months to 18 years who were given a diagnosis |
| upper respiratory infections | of upper respiratory infection (URI) and were NOT dispensed |
| Ages 3 months-18 years | an antibiotic prescription within three days of the URI |
| | diagnosis. |
| | Exclusions: |
| | Encounters with >1 diagnosis |
| | Children with a history of antibiotic Rx within 30 days |
| | of encounter |
| Follow-up care for children | Children who received an initial prescription for ADHD |
| prescribed ADHD medication | medication and: |
| Ages 6-12 years | Received at least one follow-up visit with a prescriber |
| | within 30 days of initiation of medication |
| | Remained on the medication for at least 210 days and |
| | who, in addition to the visit in the initiation phase, had at |
| | least two more follow-up visits between four weeks and |
| | nine months |

Women and adolescent girls

| | |
|---|---|
| Chlamydia screening | Women identified as presumed sexually active by pharmacy |
| Age 16-24 and sexually active | Rx data or claims data indicating potential sexual activity |
| | Screening test for chlamydia yearly |
| | Exclusions: |
| | Women who had a pregnancy test followed within seven |
| | days by either a prescription for Accutane (isotretinoin) |
| | or an X-ray. |
| Non-recommended cervical | Adolescent females 16-20 years of age who were screened |
| cancer screening in adolescent | unnecessarily for cervical cancer (lower score is better). |
| females | |
| Breast cancer screening | Mammogram in the measurement year or one year prior. |
| Age 50-74 | Exclusions: |
| | Women who have had bilateral mastectomy or two |
| | unilateral mastectomies |
| | Women 81 and older with frailty or advanced illness |
| Prenatal and | Prenatal visit during the required timeframe these timeframes |
| Postpartum care | are based on member enrollment. |
| Pregnant women | Postpartum visit to an OB/GYN or other prenatal care |
| | practitioner or PCP between 7 and 84 days after delivery. |
| Cervical cancer screening | The percentage of women 21-64 years of age who were |
| Ages 21-64 | screened for cervical cancer using either of the following |
| | criteria: |
| | Women 21-64 years of age who had cervical cytology |
| | performed within the last 3 years. |
| | Women 30-64 years of age who had cervical high-risk human |
| | papillomavirus (hrHPV) testing performed within the last 5 |
| | years. |
| | Women 30-64 years of age who had cervical cytology/high- |
| | risk human papillomavirus (hrHPV) cotesting within the last |
| | five years. |
| | Exclusions: |
| | Women who have had a complete hysterectomy with no |
| | residual cervix. |
| Prenatal (PND) and postpartum | The PND measure assesses the proportion of deliveries in |
| (PDS) depression screening and | which members were screened for clinical depression while |
| follow-Up | pregnant and if screened positive, received follow-up care. |
| | Two rates are reported. |
| | Depression Screening |
| | The proportion of deliveries in which members were |
| | screened for clinical depression using a standardized |
| | instrument during pregnancy. |
| | Follow-Up on Positive Screen |
| | The proportion of deliveries in which members received |
| | follow-up care within 30 days of screening positive for |
| | depression. |
| | The PDS measure assesses the proportion of deliveries in |
| | which members were screened for clinical depression during |
| | the postpartum period, and if screened positive, received |
| | follow-up care. Two rates are reported. |
| | Depression Screening |
| | The proportion of deliveries in which members were |
| | screened for clinical depression using a standardized |
| | instrument within 12 weeks (84 days) after delivery. |
| | Follow-Up on Positive Screen |
| | The proportion of deliveries in which members received |
| | follow-up care within 30 days of screening positive for |
| | depression. |

Adults

| | |
|---|---|
| Adults' access to | Patients 20 years and older who had an ambulatory or |
| preventive/ambulatory health | preventive care visit. |
| services | |
| Ages 20 and older | |
| Colorectal cancer screening | One or more of the following screenings: |
| Ages 50-75 | Fecal occult blood test annually |
| | FIT-DNA test every three years |
| | Flexible sigmoidoscopy every five years |
| | CT colonography every five years |
| | Colonoscopy every 10 years |
| | Exclusions: |
| | Colorectal cancer |
| | Total colectomy |
| | Members 81 and older with frailty or advanced illness or |
| | dementia |
| | Hospice and palliative care |
| Non-recommended PSA-based | Men 70 years and older who were screened for prostate cancer |
| screening in older men (PSA) | using prostate-specific antigen (PSA)-based testing (lower |
| | score is better). |
| Plan all-cause readmissions | Percentage of acute inpatient stays followed by an acute |
| | readmission for any cause within 30 days for 18 years and |
| | older. |
| Osteoporosis management in | Women who received the following within six months of |
| women who had a fracture | suffering a fracture: |
| Age 67-85 | Bone mineral density (BMD) test |
| | Prescription for a drug to treat or prevent osteoporosis in |
| | the six months after the fracture |
| | Exclusions: |
| | Women who received a BMD screening in the two years |
| | prior to the fracture. |
| | Women who received a prescription for a drug to treat or |
| | prevent osteoporosis 12 months prior to the fracture |
| | Fractures of the finger, toe, face and skull are not |
| | included in this measure. |
| | Women 81 and older with frailty or advanced illness |
| Transitions of care | The percentage of discharges for members who had each of the |
| 18 years of age or older | following during the measurement year. Four rates are |
| | reported: |
| | Notification of inpatient admission |
| | Documentation of receipt of notification of inpatient |
| | admission on the day of admission or the following day |
| | Receipt of discharge information |
| | Documentation of receipt of discharge information on the |
| | day of discharge or the following day |
| | Patient engagement after inpatient discharge |
| | Documentation of patient engagement (e.g., office visits, |
| | visits to the home, telehealth) provided within 30 days of |
| | discharge. |
| | Medication reconciliation post-discharge |
| | Documentation of medication reconciliation on the date |
| | of discharge through 30 days after discharge (31 total |
| | days). |

NaviCare

| | |
|---|---|
| Care for older adults | Adults aged 65 and older who had each of the following during |
| Age 65 years and older | the measurement year: |
| | Advance care planning |
| | Medication review |
| | Functional status assessment |
| | Pain assessment |

Alcohol and other drug dependence

| | |
|---|---|
| Initiation and engagement of | Patients diagnosed with alcohol and other drug dependence |
| alcohol and other drug (AOD) | who: |
| dependence treatment | Initiate treatment within 14 days of diagnosis |
| Ages 13 and older: | Receive two additional AOD services within 30 days of |
| 13-17 years | initiation |
| 18+ years | |
| Total | |
| Follow-up after emergency | The percentage of ED visits for which the member |
| department visit for alcohol and | received follow-up within 30 days of the ED visit. |
| other drug dependence | The percentage of ED visits for which the member |
| 13 years of age and older with a | received follow-up within seven days of the ED visit. |
| principal diagnosis of alcohol or | |
| other drug dependence | |
| Risk of continued opioid use | The percentage of members 18 years and older who have a |
| 18 years of age and older | new episode of opioid use that puts them at risk for continued |
| | opioid use. Two rates are reported: |
| | The percentage of members whose new episode of opioid |
| | use lasts at least 15 days in a 30-day period. |
| | The percentage of members whose new episode of opioid |
| | use lasts at least 31 days in a 62 day period. |
| Pharmacotherapy for opioid use | The percentage of new opioid use disorder pharmacotherapy |
| disorder | events with OUD pharmacotherapy for 180 or more days |
| | among members age 16 and older with a diagnosis of OUD. |
| Follow-up after high-intensity | The percentage of acute inpatient hospitalizations, residential |
| care for substance use disorder | treatment or detoxification visits for a diagnosis of substance |
| | use disorder among members 13 years of age and older that |
| | result in a follow-up visit or service for substance use disorder. |
| | Two rates are reported: |
| | The percentage of visits or discharges for which the |
| | member received follow-up for substance use disorder |
| | within the 30 days after the visit or discharge. |
| | The percentage of visits or discharges for which the |
| | member received follow-up for substance use disorder |
| | within the 7 days after the visit or discharge. |

Asthma

| | |
|---|---|
| Asthma medication ratio | The percentage of members 5-85 years of age who were |
| Ages 5-85 years | identified as having persistent asthma and had a ratio of |
| Age stratifications: | controller medications to total asthma medications of 0.50 or |
| 5-11 years | greater during the measurement year. |
| 12-18 years | Exclusions: Emphysema, COPD, cystic fibrosis, acute |
| 19-50 years | respiratory failure or no dispensed medications. |
| 51-64 years | |
| 65-85 years | |
| Total rate | |

Cardiac

| | |
|---|---|
| Persistence of beta-blocker | Patients who were hospitalized and discharged alive after an |
| treatment after heart attack | acute MI who: |
| Age 18 and older | Received treatment with beta-blockers for six months |
| | after discharge |
| | Exclusions: |
| | Patients identified as having a contraindication to beta- |
| | blocker therapy |
| | Patients with a history of adverse reaction to beta-blocker |
| | therapy |
| Controlling high blood pressure | Patients 18-85 with a diagnosis of hypertension whose most |
| Age 18-85 | recent blood pressure reading was controlled (<140/90 mm |
| | Hg) in the measurement year (taken by any digital device). |
| | Exclusions: |
| | Patients with end stage renal disease (ESRD) or kidney |
| | transplant |
| | Pregnant during the measurement year |
| | Admission to a non-acute inpatient setting during the |
| | measurement year |
| | Members 81 and older with frailty or advanced illness |
| | Palliative care |

Chronic obstructive pulmonary disease (COPD)

| | |
|---|---|
| Use of spirometry testing in the | Adults with a new (within the measurement year) diagnosis |
| assessment and diagnosis of | or newly active COPD who received spirometry testing to |
| COPD | confirm the diagnosis. |
| Ages 40 and older | Spirometry testing must occur 730 days prior to or 180 |
| | days after the diagnosing event. |
| Pharmacotherapy management | Adults aged 40 or older who had an acute inpatient discharge |
| of COPD exacerbation | or an ED encounter with a principal diagnosis of COPD who |
| Adults 40 and older | were dispensed both: |
| | A systemic corticosteroid within 14 days of discharge |
| | Bronchodilator within 30 days of discharge |
| | NOTE: the eligible population for this measure is based on the |
| | discharges and visits, not the patient. It is possible for the |
| | denominator for this measure to include multiple events for the |
| | same patient. |

Depression

| | |
|---|---|
| Antidepressant medication | Adults newly diagnosed with depression and treated with an |
| management | antidepressant who received the following: |
| Ages 18 years and older | Effective acute phase: filled sufficient number of Rx to |
| | allow for 84 days of continuous therapy. |
| | Effective continuation phase: filled sufficient number of |
| | Rx to allow for 180 days of continuous therapy. |
| | To qualify as a new diagnosis, two criteria must be met: |
| | A 120-day (four-month) negative diagnosis history on or |
| | before the start date |
| | A 90-day (three-month) negative medication history on |
| | or before the start date |

Diabetes

| | |
|---|---|
| Comprehensive diabetes care | Yearly screening of the following: |
| Age 18-75 | HbA1c testing |
| | HbA1c result > 9.0 = poor control |
| | HbA1c result < 8.0 = good control |
| | Retinal eye exam |
| | Monitoring for nephropathy |
| | Blood pressure reading <140/90 (taken by any digital |
| | device) |
| | Exclusions |
| | Members 81 and older with frailty or advanced illness |
| | Palliative care |
| | Polycystic ovarian syndrome (PCOS) |

Mental illness

| | |
|---|---|
| Follow-up after hospitalization | Patients discharged from an inpatient mental health admission |
| for mental illness | and received: |
| Age 6 and over | One follow-up encounter with a mental health provider |
| | within seven days after discharge. |
| | One follow-up encounter with a mental health provider |
| | within 30 days after discharge. |

Rheumatoid arthritis

| | |
|---|---|
| Anti-rheumatic drug therapy for | Patients 18 years and older who were diagnosed with |
| rheumatoid arthritis | rheumatoid arthritis and who were dispensed at least one |
| | ambulatory prescription for a disease modifying anti-rheumatic |
| | drug (DMARD). |
| | Exclusions |
| | Members 81 and older with frailty or advanced illness |

Tobacco users

| | |
|---|---|
| Medical assistance with | Current smokers who were seen by a practitioner during the |
| smoking cessation | measurement year and: |
| Tobacco users age 18 and older | Received advice to quit |
| | Cessation medications were recommended and discussed |
| | Cessation methods were recommended or discussed |
| | Information is received via CAHPS ® survey methodology. |

Other

| | |
|---|---|
| Appropriate testing for | Children who had an outpatient visit or ED encounter with |
| pharyngitis | only a diagnosis of pharyngitis who were dispensed an |
| 3 years of age and older | antibiotic and also received a Group A streptococcus test three |
| | days before or three days after the prescription. |
| | Exclusions: |
| | Encounters with >1 diagnosis |
| | Children with a history of antibiotic Rx within 30 days of |
| | encounter |
| Avoidance of antibiotic | Members diagnosed with acute bronchitis who did not receive |
| treatment for acute bronchitis | an antibiotic Rx on or within seven days of diagnosis. |
| Ages 3 months of age and older | |

What is claimed is:

1. An apparatus for resource allocation optimization, the apparatus comprising at least one processor and at least one non-transitory storage medium storing instructions that, with the at least one processor, configure the apparatus to:
receive a resource allocation optimization request, the resource allocation optimization request comprising a plan identifier and a member data structure population identifier;
retrieve a plurality of member data structures based at least in part on the member data structure population identifier;
retrieve a plurality of measure data structures based at least in part on the plan identifier;
for each measure data structure of the plurality of measure data structures,
generate a first number of benchmark points associated with a first benchmark level, wherein the first number of benchmark points is based at least in part on a first number of compliant member data structures of the plurality of member data structures available for the measure data structure;
generate a second number of benchmark points based at least in part on a second number of compliant member data structures of the plurality of member data structures required for a second benchmark level that is higher than the first benchmark level; and
generate an optimization score;
upon determining that a maximum obtainable benchmark points value meets a benchmark points value threshold, generate a third number of benchmark points representing a required number of benchmark points for an overall rating level associated with the plan identifier to increase from a current rating level to a next rating level;
generate a resource allocation optimization interface configured to render graphical representations of the plan identifier, the current rating level, the next rating level, the third number of benchmark points, and the plurality of measure data structures displayed in an order according to their respective optimization scores; and
provide the resource allocation optimization interface for display via display interface of a client computing device.

2. The apparatus of claim 1, wherein the resource allocation optimization interface is further configured to render a graphical representation of the second number of benchmark points associated with each measure data structure.

3. The apparatus of claim 2, wherein the resource allocation optimization interface is further configured to render an indication of a minimum number of measure data structures to which resources should be allocated in order to achieve the next rating level.

4. The apparatus of claim 1, wherein the resource allocation optimization request is received originating from the client computing device.

5. The apparatus of claim 1, wherein the plan identifier and member data structure population identifier are received as a result of electronic interactions with a graphical user interface by a user of the client computing device.

6. The apparatus of claim 1, wherein the current rating level is a HEDIS rating.

7. The apparatus of claim 1, wherein the next rating level is a HEDIS rating.

8. The apparatus of claim 1, wherein the optimization score is generated according to:
$$Optimization Score = \frac{Weighting * ( Max Hits - Current Hits )}{Complexity * ( \frac{Hits to Next Percentile}{Denominator - Numerator} )}$$
where Weighting represents a weighting value associated with a measure identifier of a measure data structure for which the optimization score is being generated, Max Hits represents a maximum number of compliant member data structures available for the measure identifier, Complexity represents a complexity value associated with the measure identifier, Numerator represents a first number of compliant member data structures available for the measure identifier, Denominator represents a second number of eligible member data structures of the plurality of member data structures available for the measure identifier, Current Hits represents a ratio of Numerator to Denominator, and Hits to Next Percentile represents a third number of required additional compliant member data structures in order to achieve a next percentile for the measure identifier.

9. A computer program product for resource allocation optimization, the computer program product comprising at least one non-transitory storage medium storing instructions that, with at least one processor, configure an apparatus to:
receive a resource allocation optimization request, the resource allocation optimization request comprising a plan identifier and a member data structure population identifier;
retrieve a plurality of member data structures based at least in part on the member data structure population identifier;
retrieve a plurality of measure data structures based at least in part on the plan identifier;
for each measure data structure of the plurality of measure data structures,
generate a first number of benchmark points associated with a first benchmark level, wherein the first number of benchmark points is based at least in part on a first number of compliant member data structures of the plurality of member data structures available for the measure data structure;
generate a second number of benchmark points based at least in part on a second number of compliant member data structures of the plurality of member data structures required for a second benchmark level that is higher than the first benchmark level; and
generate an optimization score;
upon determining that a maximum obtainable benchmark points value meets a benchmark points value threshold, generate a third number of benchmark points representing a required number of benchmark points for an overall rating level associated with the plan identifier to increase from a current rating level to a next rating level;
generate a resource allocation optimization interface configured to render graphical representations of the plan identifier, the current rating level, the next rating level, the third number of benchmark points, and the plurality of measure data structures displayed in an order according to their respective optimization scores; and
provide the resource allocation optimization interface for display via display interface of a client computing device.

10. The computer program product of claim 9, wherein the resource allocation optimization interface is further configured to render a graphical representation of the second number of benchmark points associated with each measure data structure.

11. The computer program product of claim 10, wherein the resource allocation optimization interface is further configured to render an indication of a minimum number of measure data structures to which resources should be allocated in order to achieve the next rating level.

12. The computer program product of claim 9, wherein the resource allocation optimization request is received originating from the client computing device.

13. The computer program product of claim 9, wherein the plan identifier and member data structure population identifier are received as a result of electronic interactions with a graphical user interface by a user of the client computing device.

14. The computer program product of claim 9, wherein the current rating level is a HEDIS rating.

15. The computer program product of claim 9, wherein the next rating level is a HEDIS rating.

16. The computer program product of claim 9, wherein the optimization score is generated according to:
$$Optimization Score = \frac{Weighting * ( Max Hits - Current Hits )}{Complexity * ( \frac{Hits to Next Percentile}{Denominator - Numerator} )}$$
where Weighting represents a weighting value associated with a measure identifier of a measure data structure for which the optimization score is being generated, Max Hits represents a maximum number of compliant member data structures available for the measure identifier, Complexity represents a complexity value associated with the measure identifier, Numerator represents a first number of compliant member data structures available for the measure identifier, Denominator represents a second number of eligible member data structures of the plurality of member data structures available for the measure identifier, Current Hits represents a ratio of Numerator to Denominator, and Hits to Next Percentile represents a third number of required additional compliant member data structures in order to achieve a next percentile for the measure identifier.

17. A computer implemented method for resource allocation optimization, the method comprising:
receiving a resource allocation optimization request, the resource allocation optimization request comprising a plan identifier and a member data structure population identifier;
retrieving a plurality of member data structures based at least in part on the member data structure population identifier;
retrieving a plurality of measure data structures based at least in part on the plan identifier;
for each measure data structure of the plurality of measure data structures,
generating a first number of benchmark points associated with a first benchmark level, wherein the first number of benchmark points is based at least in part on a first number of compliant member data structures of the plurality of member data structures available for the measure data structure;
generating a second number of benchmark points based at least in part on a second number of compliant member data structures of the plurality of member data structures required for a second benchmark level that is higher than the first benchmark level; and
generating an optimization score;
upon determining that a maximum obtainable benchmark points value meets a benchmark points value threshold, generating a third number of benchmark points representing a required number of benchmark points for an overall rating level associated with the plan identifier to increase from a current rating level to a next rating level;
generating a resource allocation optimization interface configured to render graphical representations of the plan identifier, the current rating level, the next rating level, the third number of benchmark points, and the plurality of measure data structures displayed in an order according to their respective optimization scores; and
providing the resource allocation optimization interface for display via display interface of a client computing device.

18. The method of claim 17, wherein the resource allocation optimization interface is further configured to render a graphical representation of the second number of benchmark points associated with each measure data structure.

19. The method of claim 17, wherein the resource allocation optimization interface is further configured to render an indication of a minimum number of measure data structures to which resources should be allocated in order to achieve the next rating level.

20. The method of claim 17, wherein the optimization score is generated according to:
$$Optimization Score = \frac{Weighting * ( Max Hits - Current Hits )}{Complexity * ( \frac{Hits to Next Percentile}{Denominator - Numerator} )}$$
where Weighting represents a weighting value associated with a measure identifier of a measure data structure for which the optimization score is being generated, Max Hits represents a maximum number of compliant member data structures available for the measure identifier, Complexity represents a complexity value associated with the measure identifier, Numerator represents a first number of compliant member data structures available for the measure identifier, Denominator represents a second number of eligible member data structures of the plurality of member data structures available for the measure identifier, Current Hits represents a ratio of Numerator to Denominator, and Hits to Next Percentile represents a third number of required additional compliant member data structures in order to achieve a next percentile for the measure identifier.
